(19) United States
(12) Patent Application Publication
(10) Pub. No.: US 2014/0187795 A1
(43) Pub. Date: Jul. 3, 2014

(54) CRTH2 MODULATORS AND PREPARATION THEREOF

(71) Applicants: James Jia, Somerville, MA (US); Regina Graul, Duxbury, MA (US); Wayne C Schairer, Westborough, MA (US); Vishnu Karnati, Sudbury, MA (US); Ray Forslund, Natick, MA (US)

(72) Inventors: James Jia, Somerville, MA (US); Regina Graul, Duxbury, MA (US); Wayne C Schairer, Westborough, MA (US); Vishnu Karnati, Sudbury, MA (US); Ray Forslund, Natick, MA (US)

(73) Assignee: Ironwood Pharmaceuticals, Inc., Cambridge, MA (US)

(21) Appl. No.: 13/992,099

(22) PCT Filed: Jun. 17, 2011
(86) PCT No.: PCT/US11/40870

§ 371 Date: Dec. 12, 2013

Related U.S. Application Data
(60) Provisional application No. 61/420,971, filed on Dec. 8, 2010.

(51) Int. Cl. C07D 207/34; C07D 207/48

(52) U.S. Cl. CPC C07D 207/34; C07D 207/48
USPC 548/518; 548/561; 548/542

(57) ABSTRACT

The present invention relates to processes and intermediates for the preparation of compounds useful as CRTH2 antagonists. Compounds prepared by the processes of the invention are of Formula (AI), (All) and Compound (I).

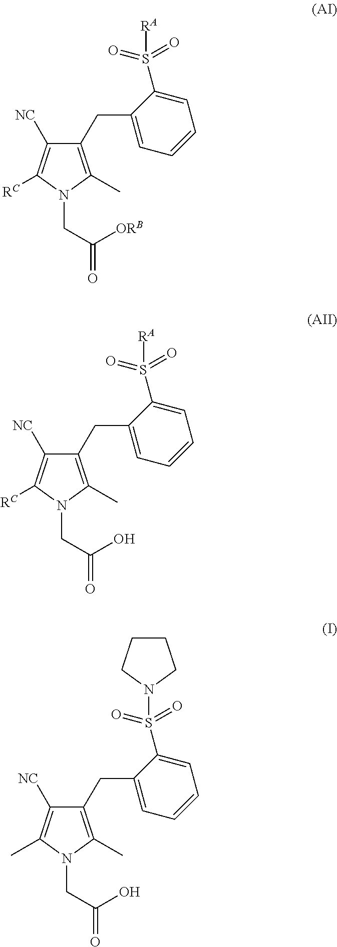

This patent application claims the benefit of U.S. Provisional Application No. 61,420,971, filed Dec. 8, 2010, the disclosure of which is herein incorporated by reference in the entirety.

### TECHNICAL FIELD

The present disclosure relates to novel processes for the preparation of compounds useful as modulators of chemoattractant receptor-homologous molecule expressed on T helper type 2 cells (CRTH2), particularly CRTH2 antagonists that are useful for treating various disorders, including asthma and allergic and respiratory disorders. These processes are amenable to large scale preparation and produce stable substituted 2-(3-cyano-5-methyl-4-(1-ylsulfonyl)benzyl)-1H-pyrrol-1-yl)acetic esters of Formula AI or acids of Formula AII in high purity and yields. This disclosure also provides novel intermediates useful in the preparation of said compounds.

### BACKGROUND

CRTH2 is a Gαᵢ protein-coupled receptor involved in both mediating PGD2-induced chemoattraction and in activation of specific cell types involved in allergic inflammation. CRTH2 is expressed by Th2 cells, eosinophils and basophils, but not by Th1 cells, B cells or NK cells. PGD2 is produced by allergen-activated mast cells and has been implicated in various allergic diseases as a pro-inflammatory mediator, such as asthma, rhinitis and allergies. Thus, blocking binding of PGD₂ to CRTH2 is a useful therapeutic strategy for treatment of such diseases.

CRTH2 agonists activate eosinophils, basophils and Th2 cells in vitro, resulting in induction of actin polymerization, calcium influx, CD11b expression and chemotaxis. Injection of a CRTH2 agonist in vivo can elicit transient recruitment of eosinophils from bone marrow into the blood. A genetic study of African American and Chinese cohorts found that polymorphisms in CRTH2 were tightly associated with asthma susceptibility. Thus, it has been suggested that modulators of CRTH2, particularly CRTH2 inhibitors, may be useful in the prevention and/or treatment of allergic asthma and other allergic disorders as recruitment and/or activation of eosinophils, basophils and Th2 cells is a prominent feature of the changes that occur in the asthmatic lung. Similar activation of these cell types, or subsets thereof, is believed to play an important role in the etiology of other diseases, including eosinophilic esophagitis and atopic dermatitis. This fact, combined with the fact that CRTH2 mediates PGD₂-induced chemotaxis, suggests that compounds that alter chemotaxis by inhibiting CRTH2 activity could be useful in controlling various diseases and disorders, including, without limitation, allergic asthma, chronic airway inflammation, atopic dermatitis, chronic obstructive pulmonary disease (COPD), and/or eosinophilic esophagitis.

Compounds that alter chemotaxis by inhibiting CRTH2 activity could also be useful in controlling allergic rhinitis, which is classified as either seasonal (SAR) or perennial (PAR) depending upon the type of trigger and duration of symptoms. SAR symptoms occur in the spring, summer and/or early fall and can be triggered by outdoor allergens such as airborne tree, grass and weed pollens while PAR is usually persistent and chronic with symptoms occurring year-round and is commonly associated with indoor allergens such as dust mites, animal dander and/or mold spores. Symptoms of allergic rhinitis may include runny nose, nasal itching, sneezing, watery eyes and nasal congestion.

CRTH2 agonists can induce desensitization of the cell system by promoting internalization and down regulation of the cell surface receptor. For example, certain CRTH2 agonists can induce desensitization of PGD₂-responsive cells to subsequent activation by a CRTH2 agonist. Therefore, CRTH2 modulators that are CRTH2 agonists may be therapeutically useful because they can cause the desensitization of PGD₂-responsive cells. Importantly, CRTH2 agonists may also cause cross-desensitization. Cross-desensitization, which can occur in many cell-signaling systems, refers to a phenomenon whereby an agonist for one receptor can reduce or eliminate sensitivity of a cell type to an unrelated agonist/receptor signaling system. For example, treatment with the CRTH2 agonist indomethacin reduces expression of CCR3, the receptor for the chemoattractant, eotaxin.

CRTH2 is also found on cell types outside the immune system, including spinal cord neurons and brain. PGD₂ activation of CRTH2, e.g., during inflammation, can lead to hyperalgesia, allodynia and neuropathic pain. Thus, inhibitors of CRTH2 may be used to treat hyperalgesia, allodynia and neuropathic pain.

2-(3-cyano-2,5-dimethyl-4-(2-(pyrrolidin-1-ylsulfonyl)benzyl)-1H-pyrrol-1-yl)acetic acid (Compound I), having the structure depicted below, is a CRTH2 antagonist that has demonstrated efficacy for the treatment of asthma and allergic rhinitis in preclinical models.

Compound I is described in U.S. Provisional Application No. 61/289,841 filed Dec. 23, 2009 (compound I-32), and also in U.S. Non-Provisional application Ser. No. 12/969,840 filed Dec. 16, 2010, the disclosures of which applications are herein incorporated by reference in their entirety.

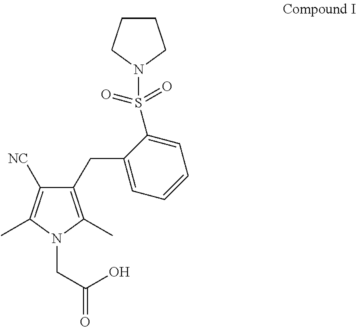 [SMILES: Cc1c(C#N)c(Cc2ccccc2S(=O)(=O)N2CCCC2)c(C)n1CC(=O)O]

Other compounds of Formula AI or Formula AII (depicted below) have been shown to be CRTH2 antagonists and are useful for the treatment of asthma and other respiratory diseases (such as allergic rhinitis). Compounds of Formula AI may also be used as pro-drugs of compounds of Formula AII or as intermediates in the synthesis of compounds of Formula AII. The compounds of Formula AI and AII are also disclosed in U.S. Provisional Application No. 61/289,841, the disclosures of which are incorporated by reference in their entirety.

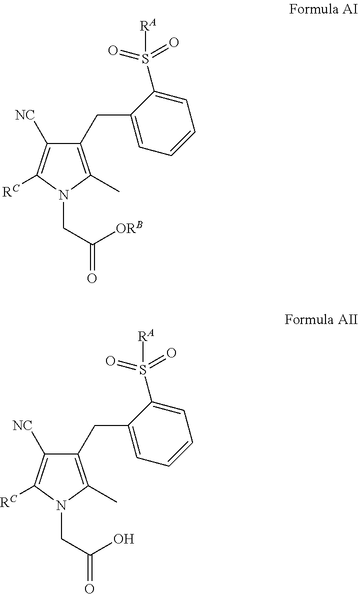

### SUMMARY OF THE INVENTION

Novel processes for preparing substituted 2-(3-cyano-5-methyl-4-(1-ylsulfonyl)benzyl)-1H-pyrrol-1-yl)acetic esters of Formula AI or acids of Formula AII are described herein. In one embodiment, a process for preparing 2-(3-cyano-2,5-dimethyl-4-(2-(pyrrolidin-1-ylsulfonyl)benzyl)-1H-pyrrol-1-yl)acetic acid (Compound I) is also described.

In one aspect, a process for making a compound of Formula AII is provided, said process comprising the steps of:
i) conducting reductive alkylation of a pyrrole starting material 2A with an aldehyde starting material 3A, in the presence of a Lewis acid and a reducing agent, in an aprotic organic solvent, to afford an ester of Formula AI,

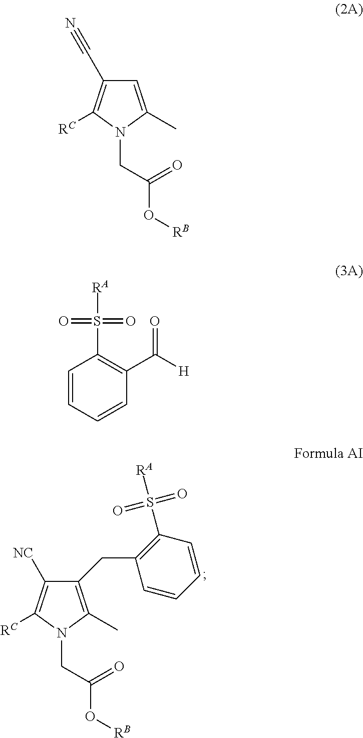

and
ii) saponifying the ester moiety of the ester of Formula AI in a solvent system, in the presence of an alkali metal hydroxide, affording, after treatment with an aqueous mineral acid, an acid of Formula AII,

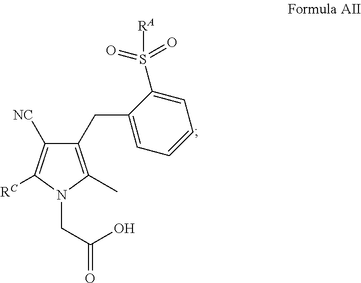

wherein:

R^{A} is selected from phenyl or an N-linked 5 or 6-membered heterocycle, optionally containing up to two ring heteroatoms selected from O, N or S in addition to the N ring atom of the 5 or 6-membered heterocycle linked to the sulfur atom of the sulfonyl group; wherein, the linked N ring atom is directly attached to the sulfur atom of the sulfonyl group to form a sulfonamide;

R^{B} is a C₁₋₆ alkyl; and

R^{C} is selected from C₁₋₆ alkyl, a 3 to 6-membered cycloalkyl ring or phenyl, in said intermediates of Formulae 2A and 3A and said compounds of Formulae AI and AII.

In another aspect, a process for preparing Compound I is provided; said process comprising the steps of:

i) reductively alkylating a pyrrole starting material 2 with an aldehyde starting material 3, in the presence of a Lewis acid and a reducing agent, in an aprotic organic solvent, to afford an ester of Formula 4,

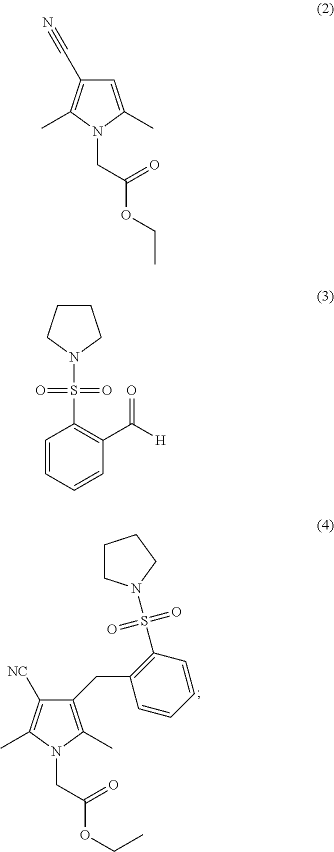 [SMILES: CCOC(=O)Cn1c(C)cc(C#N)c1C ; O=Cc1ccccc1S(=O)(=O)N1CCCC1 ; CCOC(=O)Cn1c(C)c(C#N)c(Cc2ccccc2S(=O)(=O)N2CCCC2)c1C]

and

ii) saponifying the ester moiety of the ester of Formula 4 in a solvent system, in the presence of an alkali metal hydroxide, affording, after treatment with an aqueous mineral acid, the acid Compound I,

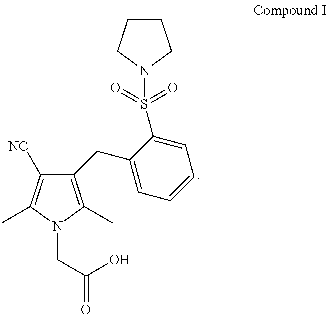 [SMILES: Cc1c(C#N)c(Cc2ccccc2S(=O)(=O)N2CCCC2)c(C)n1CC(=O)O]

Novel intermediates that are useful in the processes herein described are also described as well as methods for preparing said intermediates.

The present invention also includes the use of the intermediates, e.g., compounds of Formula IA or IA′, compounds of Formula 2A or pyrrole starting material 2A, starting material 2, compounds of Formula 2A′, compounds of Formula 3A or aldehyde starting material 3A, starting material 3, the compound of Formula 4, compounds of Formula 5A, the compound of Formula 5, the compound of Formula 6, compounds of Formula 8A, the compound of Formula 8, compounds of Formula 9A, and/or the compound of Formula 9, disclosed herein to prepare the compounds of Formula AI or AII, or Compound I.

The present invention is also directed to the compounds of Formula AI or Formula AII, and Compound I, and pharmaceutically acceptable salts thereof.

### DETAILED DESCRIPTION OF THE INVENTION

Reference will now be made in detail to certain embodiments of the invention, examples of which are illustrated in the accompanying schemes, structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

The phrase “optionally substituted” is used interchangeably with the phrase “substituted or unsubstituted.” In general, the term “substituted” refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As will be apparent to one of ordinary skill in the art, groups such as —H, halogen, —NO₂, —CN, —OH, —NH₂ or —OCF₃ would not be substitutable groups.

The phrase “up to”, as used herein and referring to compound substitution, refers to zero or any integer number that is equal or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. It will be understood by one of ordinary skill in the art that when a group is characterized as substituted (as opposed to optionally substituted) with, e.g., “up to 3” substituents, it can only be substituted with 1, 2 or 3 substituents.

When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary skill in the art and may be determined without undue experimentation. The term“stable”, as used herein, refers to compounds that are not substantially altered when subjected to conditions that allow for their production, detection, and, in some embodiments, their recovery, purification, and use for one or more of the purposes disclosed herein.

In some embodiments, a stable compound or chemically feasible compound is one that is not substantially altered when kept at a temperature of +25° C. or less, in the absence of moisture or other chemically reactive conditions, for at least a week.

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Heterocycle” (or “heterocyclyl” or “heterocyclic), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”. In some instances the term can be used in the phrase “aromatic heterocycle”, and in this case it refers to a “heteroaryl group” as defined below. The term heterocycle also includes fused, spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is on the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads”. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually a quaternary carbon atom).

The term “ring atom” refers to an atom such as C, N, O or S that is part of the ring of an aromatic group, a cycloaliphatic group or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of R^{o} as in Formula D1:

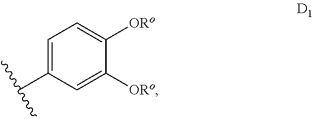

these two occurrences of R^{o} are taken together with the oxygen atoms to which they are bound to form a fused 6-membered oxygen containing ring as in Formula D2:

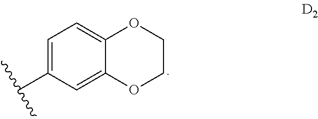 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In general, the term “vicinal” refers to the placement of substituents on a group that includes two or more carbon atoms, wherein the substituents are attached to adjacent carbon atoms.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

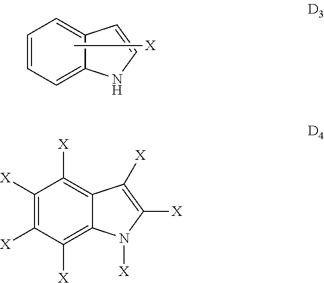

This also applies to multiple ring systems fused to optional ring systems (which would be represented by dotted lines). For example, in Formula D5, X is an optional substituent both for ring A and ring B.

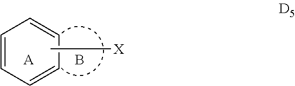

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

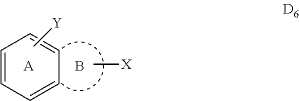

As used herein, the terms “alkoxy” or “alkylthio” refer to an alkyl group, as previously defined, attached to the molecule, or to another chain or ring, through an oxygen (“alkoxy” i.e, —O-alkyl) or a sulfur (“alkylthio” i.e., —S-alkyl) atom.

The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, —CH═CHOCH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, “benzyloxy” e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or an aliphatic will be indicated separately. Thus, a 5-6-membered aryloxy(C₁₋₄alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or —C≡N.

The term “hydroxyl” or “hydroxy” refers to —OH.

A “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be _{C2} alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a _{C1-6} aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moietes. Cyclic groups can also form linkers: e.g. a 1,6-cyclohexanediyl can be a linker between two R groups, as in

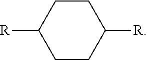

A linker can additionally be optionally substituted in any portion or position.

Divalent groups of the type R—CH═ or R₂C═, wherein both free valences are in the same atom and are attached to the same substituent, are also possible. In this case, they will be referred to by their IUPAC accepted names. For instance an alkylidene (such as, for example, a methylidene (═CH₂) or an ethylidene (e.g., ═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W. et al., “Protective Groups in Organic Synthesis”, Third Edition, John Wiley & Sons, New York: 1999, the entire contents of which is hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-ethyl-3-(3-dimethylaminopropyl)carbodiimide), DCC (dicyclohexylcarbodiimide), BOP (benzotriazol-1-yloxy-tris(dimethylamino)-phosphonium hexafluorophosphate), pyBOP ((benzotriazol-1-yloxy)tripyrrolidinophosphonium hexafluorophosphate), 2,4,6-tripropyl-1,3,5,2,4,6-trioxatriphosphorinane-2,4,6-trioxide (T₃P), etc.

As used herein, when referring to a temperature range or a range of number of equivalents of a reagent or reactant, the term “between” includes any numerical value between the two limits given and includes those two limits. For example a temperature of between +5° C. and +20° C., includes any temperature value in that range (e.g. +10° C., +15° C., +13.8° C.), including also exactly +5° C. and exactly +20° C. Similarly, a number of equivalents of between 1.2 and 1.5, could be any number between the two given limits (e.g. 1.3 and 1.45), including exactly 1.2 and exactly 1.5 equivalents.

In one embodiment, a process for making a compound of Formula AII is provided, said process comprising the steps of:
i) reductively alkylating a pyrrole starting material of Formula 2A with an aldehyde starting material of Formula 3A, in the presence of a Lewis acid and a reducing agent, in an aprotic organic solvent, to afford an ester of Formula AI,

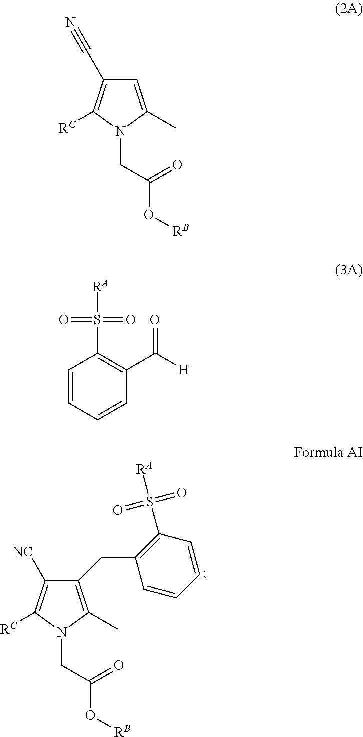

and
ii) saponifying the ester moiety of the ester of Formula AI in a solvent system, in the presence of an alkali metal hydroxide, affording, after treatment with an aqueous mineral acid, an acid of Formula AII,

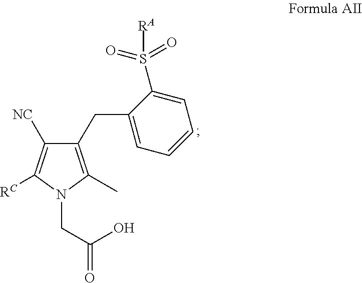

wherein:

R^{A} is selected from phenyl or an N-linked 5 or 6-membered heterocycle, optionally containing up to two ring heteroatoms selected from O, N or S, in addition to the N ring atom linked to the sulfur atom of the sulfonyl group; wherein the linked N is directly attached to the sulfur atom of the sulfonyl group to form a sulfonamide;

R^{B} is a C₁₋₆ alkyl; and

R^{C} is selected from C₁₋₆ alkyl, a 3 to 6-membered cycloalkyl ring or phenyl, in said intermediates of Formulae 2A and 3A and said compounds of Formula AI and Formula AII.

In another embodiment, a process for preparing Compound I (which is a compound of Formula AII, wherein R^{A} is pyrrolidin-1-yl and R^{C} is methyl) is provided; said process comprising the steps of:

i) reductively alkylating a pyrrole starting material 2 (which is a compound of Formula 2A, wherein R^{B} is ethyl and R^{C} is methyl) with an aldehyde starting material 3 (which is a compound of Formula 3A, wherein R^{A} is pyrrolidin-1-yl), in the presence of a Lewis acid and a reducing agent, in an aprotic organic solvent, to afford an ester of Formula 4 (which is a compound of Formula AI, wherein R^{A} is pyrrolidin-1-yl, R^{B} is ethyl and R^{C} is methyl),

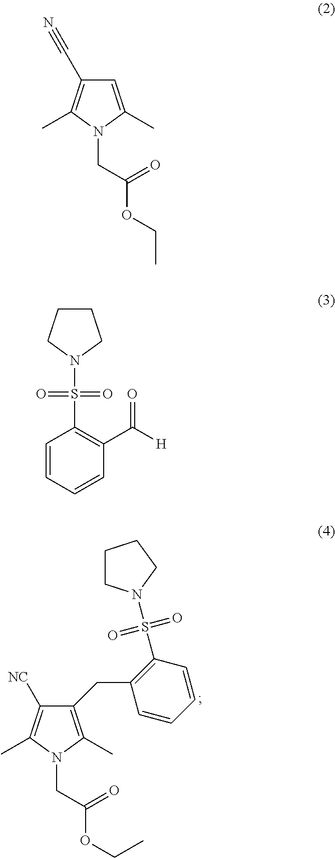 [SMILES: CCOC(=O)Cn1c(C)cc(C#N)c1C ; O=Cc1ccccc1S(=O)(=O)N1CCCC1 ; CCOC(=O)Cn1c(C)c(C#N)c(Cc2ccccc2S(=O)(=O)N2CCCC2)c1C]

and

ii) saponifying the ester moiety of the ester of Formula 4 in a solvent system, in the presence of an alkali metal hydroxide affording, after treatment with an aqueous mineral acid, the acid Compound I,

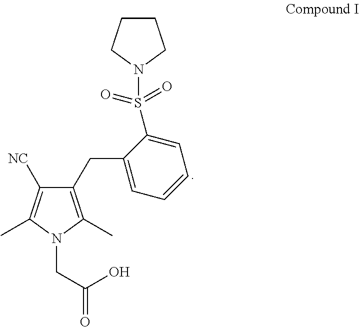 [SMILES: Cc1c(C#N)c(Cc2ccccc2S(=O)(=O)N2CCCC2)c(C)n1CC(=O)O]

In some of the embodiments of the above processes, for intermediates of Formula 3A and compounds of Formula AI and Formula AII, R^{A} is selected from phenyl or an N-linked 5 or 6-membered heterocycle, optionally containing up to two ring heteroatoms selected from O, N or S, in addition to the N ring atom linked to the sulfur atom of the sulfonyl group; wherein, the linked N is directly attached to the sulfur atom of the sulfonyl group to form a sulfonamide. In other embodiments, R^{A} is an N-linked 5 or 6-membered heterocycle, optionally containing up to two ring heteroatoms selected from O, N or S, in addition to the N ring atom linked to the sulfur atom of the sulfonyl group forming a sulfonamide. In still other embodiments, R^{A} is an N-linked pyrrolidine ring (pyrrodinyl-1-yl), wherein the nitrogen is directly linked to the sulfur atom of the sulfonyl group to give a sulfonamide.

In some of the embodiments of the above processes, R^{B} is a C₁₋₆ alkyl in said intermediates of Formula 2A and said compounds of Formula AI and Formula AII. In other embodiments, R^{B} is selected from methyl or ethyl. In still other embodiments, R^{B} is ethyl.

In some of the embodiments of the above processes, R^{C} is selected from C₁₋₆ alkyl, a 3 to 6-membered cycloalkyl ring or phenyl, in said intermediates of Formula 3A and said compounds of Formula AI and Formula AII. In other embodiments, R^{C} is selected from methyl or ethyl. In still other embodiments, R^{C} is methyl in said intermediates of Formula 3A and said compounds of Formula AI and Formula AII.

In some of the embodiments of the above processes, the compound of Formula AII is Compound I.

In some of the embodiments of the above processes, the aprotic organic solvent used in step i) is selected from acetonitrile, dichloromethane or toluene. In other embodiments, the aprotic organic solvent used in step i) is selected from acetonitrile or dichloromethane. In still other embodiments, the aprotic organic solvent used in step i) is dichloromethane.

In some of the embodiments of the above processes, the temperature used in step i) is between about −28° C. and about 0° C., e.g., between −28° C. and 0° C. In other embodiments, the temperature used in step i) is between about −28° C. and about −3° C., e.g., between −28° C. and −3° C.

In some of the embodiments of the above processes, the Lewis acid used in step i) is selected from trifluoromethylsulfonyltrimethylsilane, boron trifluoride etherate or boron trifluoride acetonitrile complex in solution. In other embodiments, the Lewis acid used in step i) is trifluoromethylsulfonyltrimethylsilane.

In some of the embodiments of the above processes, the reducing agent used in step i) is a silane. In other embodiments, said silane is triethylsilane.

In some of the embodiments of the above processes, the number of equivalents of Lewis acid per equivalent of aldehyde starting material 3A (e.g., aldehyde starting material 3) used in step i) is at least about 1.8 equivalents, e.g., 1.8 equivalents or higher, of Lewis acid per equiv of the aldehyde starting material 3A (e.g., aldehyde starting material 3). In some embodiments, the number of equivalents used is at least about 2.0 equiv, e.g., 2.0 equivalents or higher, of Lewis acid per equiv of the aldehyde starting material 3A (e.g., aldehyde starting material 3).

In some of the embodiments of the above processes, the number of equivalents of silane per equivalent of the aldehyde starting material 3A (e.g., aldehyde starting material 3) used in step i) is at least about 1.8 equivalents, e.g., 1.8 equivalents or higher, of silane per equiv of the aldehyde starting material 3A (e.g., aldehyde starting material 3). In other embodiments, the number of equivalents used is at least about 2.0 equiv, e.g., 2.0 equivalents or higher, of silane per equiv of the aldehyde starting material 3A (e.g., aldehyde starting material 3). In still other embodiments, the number of equivalents used is at least about 2.5 equiv, e.g., 2.5 equivalents or higher, of silane per equiv of the aldehyde starting material 3A (e.g., aldehyde starting material 3).

In some of the embodiments of the above processes, the isolation of the compound of Formula AI (e.g., the compound of Formula 4) at the end of step i) comprises the steps of solvent evaporation and precipitation, and is optionally followed by recrystallization.

In some of the embodiments of the above processes, said alkali metal hydroxide used in step ii) is selected from sodium hydroxide, potassium hydroxide or lithium hydroxide. In other embodiments, the alkali metal hydroxide used in step ii) is lithium hydroxide.

In some of the embodiments of the above processes, for the saponification reaction in step ii), the solvent system is comprised of a mixture of water and an aprotic solvent. In some embodiments, the aprotic solvent used in step ii) is selected from methyl t-butyl ether (MTBE), tetrahydrofuran, 2-methyltetrahydrofuran, dimethoxyethane (DME) or dioxane. In other embodiments, said aprotic solvent used in step ii) is methy t-butyl ether.

In other embodiments of the above processes, the solvent system used in step ii) additionally comprises an alcohol. In some embodiments, said alcohol is MeOH.

In some of the embodiments of the above processes, the number of equivalents of alkali metal hydroxide used in step ii) is at least 1.0 equivalents of alkali metal hydroxide per equivalent of compound of Formula AI (e.g., the compound of Formula 4). In other embodiments, the number of equivalents of alkali metal hydroxide used in step ii) is at least 1.1 equiv base per equiv of the compound of Formula AI (e.g., the compound of Formula 4). In still other embodiments, the number of equivalents of alkali metal hydroxide used in step ii) is about 1.5 equiv (e.g., 1.5 equivalents) of base per equiv of the compound of Formula AI (e.g., the compound of Formula 4).

In some of the embodiments of the above processes, the saponification step ii) is run at reflux temperature.

In some of the embodiments of the above processes, the compound of Formula AII (e.g., Compound I) is isolated at the end of step ii) by a process comprising the steps of: evaporation and precipitation, optionally followed by recrystallization. In some embodiments, the precipitation step involves the use of a mineral acid. In other embodiments, the mineral acid is an aqueous acid. In still other embodiments, the acid is selected from hydrochloric acid, sulfuric acid or phosphoric acid. In yet other embodiments, the acid is hydrochloric acid. In further embodiments the acid used is 1N HCl.

### Intermediates

In some of the embodiments, the above processes comprise the additional steps of synthesizing the pyrrole starting material 2A (e.g., 2, wherein R^{C} is methyl and R^{B} is ethyl) and the aldehyde starting material 3A (e.g., 3, wherein R^{A} is an N-linked pyrrolidine ring).

In one of the aspects (discussed in the previous section), the invention provides a process for preparing a compound of Formula AI or a compound of Formula AII as outlined in Scheme I.

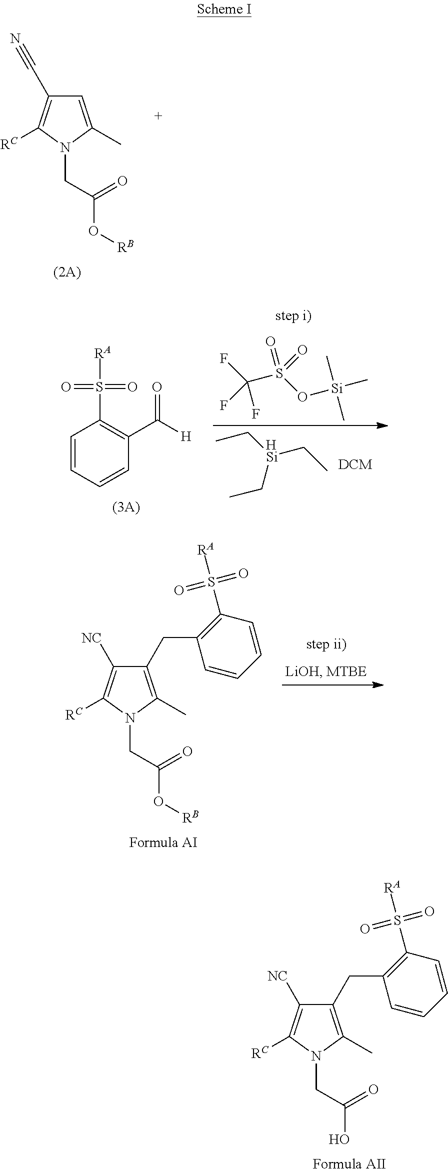

In one embodiment of the process outlined in Scheme I, the starting material of Formula 2A can be prepared according to Scheme IIa below; wherein the variables R^{A}, R^{B} and R^{C} are as defined above.

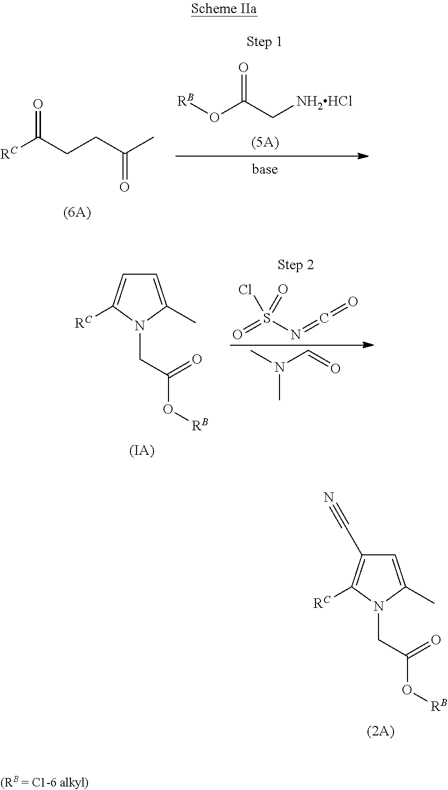

In some of the embodiments of the process outlined in Scheme IIa, the glycine ester hydrochloride intermediate of Formula 5A can be purchased commercially (e.g., the ethyl ester, wherein R^{B} is ethyl, the compound of Formula 5, can be purchased from Alfa).

In some of the embodiments of the process summarized in Scheme IIa, the intermediate of Formula 6A can be purchased commercially (e.g. the starting material 2,5-diketohexane (compound of Formula 6) can be purchased commercially, e.g., from Alfa).

In other embodiments of the process summarized in Scheme IIa, the intermediate of Formula 6A can be prepared by reacting buten-3-en-2-one with an aldehyde of general Formula R^{C}—CHO, in a solvent (e.g., water/acetone or ethanol), in the presence of a base (e.g., sodium hydroxide or triethylamine), optionally in the presence of 3-ethyl-5-(2-hydroxyethyl)-4-methylthiazol-3-ium bromide, with heating, followed by concentration under vacuum or precipitation and extraction with an organic solvent (e.g., ethyl acetate), separation of the organic layer, drying, filtration and concentrating again; and finally purifying the resulting residue by column chromatography.

In some of the embodiments of the process outlined in Scheme IIa, the intermediate of Formula 1A can be prepared by reacting a diketone of Formula 6A with a glycine ester hydrochloride intermediate of Formula 5A, in a solvent (e.g, dichloromethane or ethanol) in the presence of a base (e.g., triethylamine or solid sodium bicarbonate), optionally with heating, followed by cooling, filtration, extraction with dichloromethane, drying, filtration and concentration in vacuum; and finally purifying the resulting residue by distillation or column chromatography. In some embodiments, the base used for the preparation of the compound of Formula 1A is solid sodium bicarbonate. In other embodiments said base is triethylamine.

In one specific embodiment of the process summarized in Scheme IIa, R^{C} is methyl, and the process is outlined in Scheme IIb,

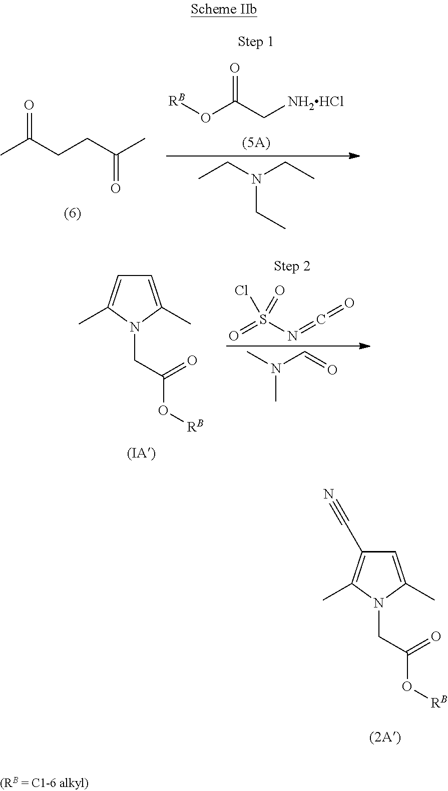

In some of the embodiments of the process summarized in Scheme IIb, the intermediate of Formula 1A′ (i.e. intermediate of Formula 1A, wherein R^{C} is methyl) can be purchased commercially (e.g., the methyl and ethyl esters are available from Aurora Fine Chemicals LLC, USA). In other embodiments, the intermediate of Formula 1A′, wherein R^{B} is methyl or ethyl, can be prepared by the procedure described in Chem. Berichte, 1953, 86, 1383-1388, herein incorporated by reference in its entirety. Alternatively, the intermediate of Formula 1A′ can be prepared as described in the EXAMPLES section of the instant application.

In one particular embodiment of Scheme IIb, the intermediate of Formula 1A′ wherein R^{B} is ethyl (i.e., compound of Formula 1), can be prepared by reacting a diketone of Formula 6 with a glycine ester hydrochloride intermediate of Formula 5A, wherein R^{B} is ethyl (i.e., compound of Formula 5), in toluene in the presence of a base (e.g., triethylamine), with heating, followed by cooling, acidification with an aqueous mineral acid (e.g. HCl), extraction into toluene, concentration under vacuum and final purification by distillation.

Referring to Scheme IIa or Scheme IIb, in step 2, the intermediate of Formula 1A or Formula 1A′ is then cyanated with chlorosulfonyl isocyanate in a non-protic solvent (e.g., DMF) to furnish an intermediate nitrile pyrrole of Formula 2A or Formula 2A′ (i.e., a compound of Formula 2A, wherein R^{C} is methyl). It should be noted that although DMF is shown in step 2 of Scheme II above, other non-protic solvents such as polar non-protic solvents can be also used.

In one embodiment, the process for the preparation of intermediate 2 from compound 6 can be carried out without the isolation of intermediate 1, as outlined in Scheme IIc; said process comprising the following steps:

A) reacting a glycine ester hydrochloride of Formula 5 and a diketone of Formula 6 in toluene, in the presence of an organic base at reflux;

B) quenching the product of step A) with an aqueous mineral acid;

C) separating the organic layer of the product of step B);

D) washing the organic layer with water;

E) concentrating the washed organic layer to give a crude oil containing compound 1; and

F) dissolving compound 1 in the crude oil obtained in step E) in DMF without any purification or isolation of compound 1, cooling the DMF solution, and reacting compound 1 with chlorosulfonyl isocyanate dissolved acetonitrile to obtain intermediate 2. Optionally, the process further comprises:

G) quenching the product of step F) with brine, followed by water, while controlling the temperature between −5° C. and 10° C.;

H) filtering off the solids from the product of step G);

I) washing the filtered solids with water; and

J) drying the washed solids, e.g., with heat such as being heated in an oven for several hours, to furnish the product of Formula 2 as a white solid in high purity.

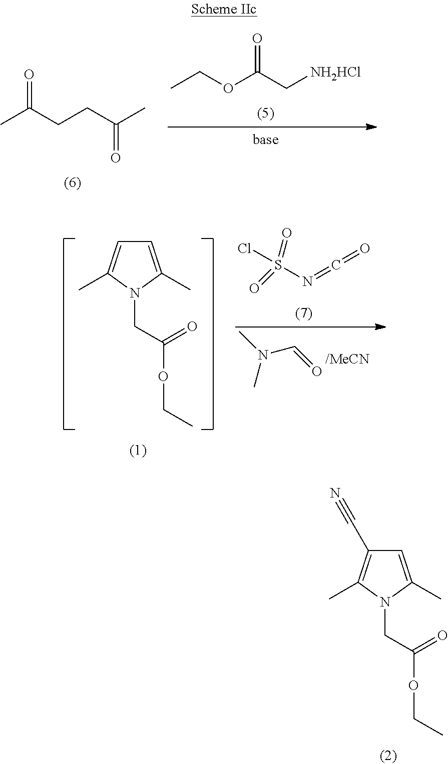

In some of the embodiments of the process outlined in Scheme IIc, the organic base used in step A) is selected from triethylamine, diethylamine, Huenigs base, pyridine, or diisopropylethylamine. In other embodiments, the base used in step A) is triethylamine.

In some of the embodiments of the process outlined in Scheme IIc, the aqueous mineral acid used in step B) is a diluted aqueous acid. In other embodiments, the aqueous acid used is selected from hydrochloric acid, phosphoric acid or sulfuric acid. In still other embodiments it is HCl. In further embodiments, it is 0.1N N HCl.

In one embodiment of the process summarized in Scheme I, for intermediates of Formula 3A, R^{A} is selected from phenyl or an N-linked 5 or 6-membered heterocycle, optionally containing up to two other ring heteroatoms selected from O, N or S, in addition to the ring N atom of the N-link; wherein, the linked N is directly attached to the sulfur atom of the sulfonyl group to form a sulfonamide. In other embodiments, R^{A} is an N-linked 5 or 6-membered heterocycle, optionally containing up to two other ring heteroatoms selected from O, N or S, in addition to the ring N atom of the N-link. In still other embodiments, R^{A} is an N-linked pyrrolidine ring, wherein the nitrogen is directly linked to the sulfur atom of the sulfonyl group to give a sulfonamide.

In some of the embodiments of the process summarized in Scheme I, the intermediate of Formula 3A, can be synthesized as described in patent application publication WO20010039982 (international patent application No. PCT/US2009/059265), published on 8 Apr. 2010, herein included by reference in its entirety.

In other embodiments of the process summarized in Scheme I, wherein R^{A} is selected from N-linked pyrrolidinyl, N-linked morpholinyl or phenyl, the intermediate of Formula 3A can be prepared by the process summarized in Scheme III.

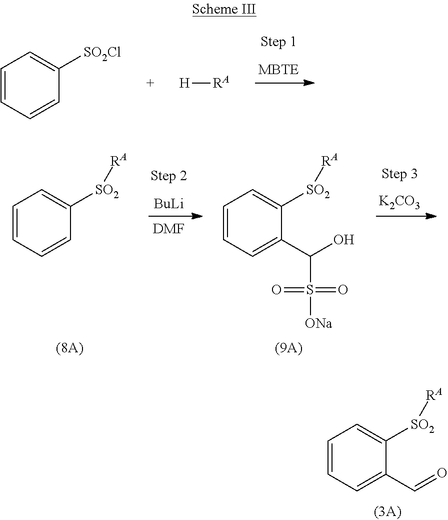

In one embodiment of the process summarized in Scheme III, the intermediate of Formula 3A is compound 3 and compound 3 can be synthesized by the process summarized in Scheme IV, which is one of the embodiments of the process of Scheme III, wherein R^{A} is pyrrolidin-1-yl.

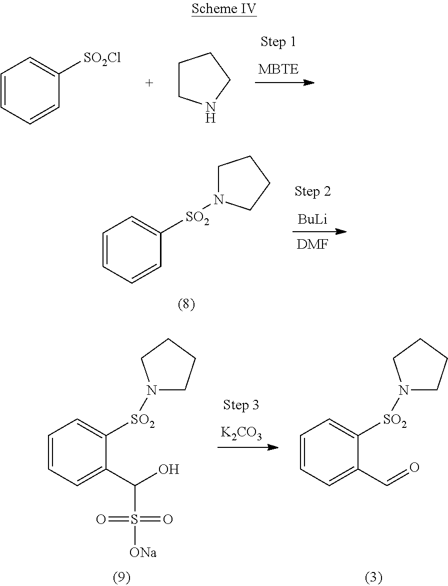

In another embodiment, the process for the synthesis of compound 3 comprises the steps of:

I) reacting benzylsulfonyl chloride with pyrrolidine, in an aprotic organic solvent to furnish the intermediate of Formula 8;
II) generating a carbanion alpha to the sulfonyl group of the phenyl ring of the intermediate of Formula 8, by using a strong organic base, in an aprotic organic solvent, at a low temperature between −78° C. and 0° C., and quenching said carbanion with a carbaldehyde donor, to furnish an intermediate hydroxy(2-(pyrrolidin-1-ylsulfonyl)phenyl)methanesulfonate of Formula 9; and
III) treating intermediate 9 with an inorganic base, in a solvent system to provide the aldehyde intermediate 3.

In one embodiment of the process for the synthesis of compound 3, summarized in Scheme IV, the aprotic organic solvent used in step I) can be at least one aprotic polar solvent. In further embodiments of the process summarized in Scheme IV, the aprotic organic solvent is selected from: tetrahydrofuran, methyl tert-butyl ether, dioxane, acetone, 2-butanone, dichloromethane, dichloroethane or chloroform. In other embodiments, the aprotic organic solvent is selected from acetone, 2-butanone or methyl tert-butyl ether. In still other embodiments, the aprotic organic solvent is methyl tert-butyl ether.

In some of the embodiments of the process for the synthesis of compound 3, the strong organic base used in step II) is an organic base at least as strong as n-BuLi, s-BuLi, t-BuLi, iPrMgCl, LDA or LiHMDS. In some embodiments, the strong organic base is selected from n-BuLi, s-BuLi, t-BuLi, iPrMgCl, LDA or LiHMDS. In further embodiments, the strong organic base is selected from n-BuLi or iPrMgCl. In still other embodiments, the strong organic base is n-BuLi.

In some of the embodiments of the process for the synthesis of compound 3, the aprotic organic solvent used in step II) is at least one aprotic polar organic solvent. In some embodiments of the process summarized in Scheme IV, the aprotic organic solvent used in step II) is selected from: THF, dioxane, N,N-dimethylformamide DMF, N,N-dimethylacetamide (DMA), N-methylpyrrolidone (NMP) or 1,3-dimethyl-2-imidazolinone (DMI). In other embodiments, the aprotic organic solvent used in step II) is DMF. In other embodiments, the aprotic organic solvent used in step II) is selected from THF or dioxane. In still other embodiments, the aprotic organic solvent used in step II) is THF.

In some of the embodiments of the process for the synthesis of compound 3, the low temperature used in step II) is between −78° C. and below 0° C. In further embodiments of the process for the synthesis of compound 3, the low temperature used in Step II) is between −15° C. and −5° C.

In some of the embodiments of the process for the synthesis of compound 3, the carbaldehyde donor used in step II) is selected from DMF or morpholine carbaldehyde. In other embodiments it is DMF.

In some of the embodiments of the process summarized in Scheme IV, the inorganic base used in step III) is selected from: sodium carbonate or potassium carbonate. In some embodiments, it is potassium carbonate.

In some of the embodiments of the process summarized in Scheme IV, the solvent system used in step III) comprises a mixture of water and an organic solvent. In some embodiments, said organic solvent is selected from: tetrahydrofuran, dioxane, acetone, 2-butanone, or t-butyl methyl ether. In other embodiments, the organic solvent is tetrahydrofuran. In still other embodiments, the solvent used in step III) is water alone.

### EXAMPLES

As used herein, abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997, herein incorporated in its entirety by reference. The following preparative examples are set forth in order that this invention is more fully understood. These examples are for the purpose of illustration only and are not to be construed as limiting the scope of the invention in any way.

### List of Common Abbreviations

### d Density DMA N,N-dimethylacetamide

### DCM Dichloromethane NMP N-methylpyrrolidone

DMF N,N-dimethylformamide DMI 1,3-dimethyl-2-imidazolinone
DME Dimethoxyethane MW Molecular Weight (see also FW)
DMSO Dimethylsulfoxide n-BuLi n-Butyl lithium
Equiv Equivalents s-BuLi sec-Buthyl lithium
Et Ethyl t-BuLi tert-Butyl lithium

### EtOAc Ethyl Acetate nm Nanometer

FW Formula Weight (see also MW) rt Room Temperature
g Gram RT Retention time
h, hrs hour or hours RTT Relative retention time
kg Kilogram THF tetrahydrofuran
L Liters MTBE methyl tert-butyl ether
min minute(s) vol Volumes

### mL Milliliters wt Weight

Mol moles

### General Analytical Techniques

HPLC was used for purity determination and for reaction monitoring. The following table describes the detailed conditions of the HPLC system and method used.

| Column: | Phenomenex Kinetex 2.6u PFP 3.0 × 10 mm, | | |
|---|---|---|---|
| | 100A | | |
| Mobile Phase: | A: 0.1% trifloroacetic acid in water | | |
| | B: 0.1% trifloroacetic acid in acetonitrile | | |
| | Time (min) | % A | % B |
| Gradient: | 0.0 | 72 | 28 |
| | 3.0 | 72 | 28 |
| | 4.5 | 40 | 60 |
| | 7.0 | 10 | 90 |
| | 8.0 | 10 | 90 |
| | 8.5 | 72 | 28 |
| | 11.0 | 72 | 28 |

| | |
|---|---|
| Flow Rate: | 0.8 mL/min |
| Total Run Time: | 11 min |
| Column Temperature: | 45° C. |
| Autosampler | n/a |
| Temperature: | |
| Detection: | 232 nm, UV absorbance |
| Injection Volume: | 10 uL |
| Analytical | 0.5 mg/mL in 50:50 water:acetonitrile (v/v) |
| Concentration: | |

### Example 1

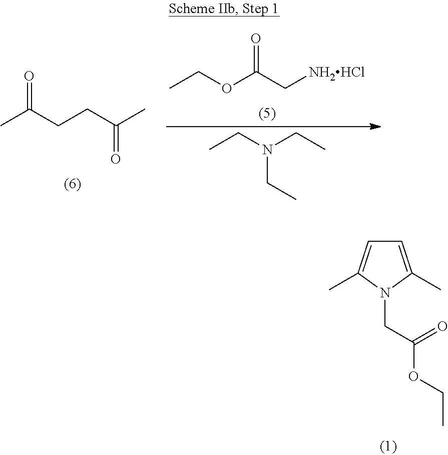

### Method 1: Using DCM as the Solvent

Hexane-2,5-dione (6, from Alfa (B25686), 214 g, 220 mL, 1875 mmol, 1.00 eq) was charged to a 5 L jacketed reactor equipped with a mechanical stirrer, an addition funnel and a thermometer, under a nitrogen purge, and was dissolved in DCM (1.5 L, 7 vol) at room temperature. Then, ethyl 2-aminoacetate hydrochloride (5, from Alfa (A10315), 314 g, 2250 mmol, 1.2 eq) was added. The mixture was cooled to 2° C. and triethylamine (569 g, 784 mL, 5625 mmol, 3 eq) was added drop-wise via an addition funnel, under a N₂ atmosphere and at such a rate as to keep the temperature between +2 to +5° C. The reaction was warmed to +20° C. over 1 h and stirred for an additional 2 h at this temperature. The reaction mixture was cooled to +5° C. and the resulting thick yellow slurry was filtered off. The filter cake was washed with DCM (1×1 L) and the combined organics were washed twice with water (2×1 L), dried over Na₂SO₄ and concentrated to dryness under reduced pressure (using a rotavaporator). The crude product (332 g) was obtained as a yellow, dense oil. This crude material was subjected to distillation to give two fractions. Fraction 1 was collected between +100 and +110° C. (oil bath temp), <92° C. distillate temperature, 0 mbar vacuum, to give 24 g of a colorless oil. It contained a ˜1:1 mixture of starting material and product. Fraction 2 was collected between +120 and +135° C. (oil bath temp), +96° C. to +100° C. distillate temperature, 0 mbar vacuum, to provide 293 g of pyrrole acetic acid ester 1 as a light yellow dense oil. This fraction contained <2% of starting material (hexane-2,5-dione) by ¹H-NMR (86% yield). The purity, as determined by HPLC was 96.5%.

¹H-NMR (400 MHz, CDCl₃) δ 1.28 (t, J=7.2 Hz, 3H), 2.17 (s, 6H), 4.22 (q, J=7.2 Hz, 2H), 4.48 (s, 2H), 5.8 (s, 2H).

HPLC Retention Times (RT) and Relative Retention Times (RRT)

| Peak | Compound | RT (min.) | RRT |
|---|---|---|---|
| 1 | — | 2.58 | 0.62 |
| 2 | 1 | 4.14 | 1.00 |

### Method 2: Using Toluene as the Solvent

Note: All equivalents and volumes are relative to ethyl 2-aminoacetate hydrochloride. Hexane-2,5-dione (6, 82 g, 84 mL, 716 mmol, 1.00 eq), ethyl 2-aminoacetate hydrochloride (5, 100 g, 716 mmol, 1.00 eq), toluene (600 mL, 6 volumes) and triethylamine (72.5 g, 100 mL, 716 mmol, 1.00 eq) were charged to a 2 L multineck round bottom flask equipped with a mechanical stirrer, a thermometer, a Dean-Stark apparatus and a condenser at room temperature. The reaction mixture was refluxed around 115° C. for 5 hours. The reaction mixture was allowed to cool to room temperature and the TLC check confirmed completion of the reaction (Hexane:EtOAc, 2:1, KMnO₄ stain, Rf=0.7). 0.1 N HCl (200 mL, 2 volumes) was added to the reaction mixture and the mixture stirred for 5 mins to dissolve all the salts. The reaction mixture was then transferred to a separating funnel and the aqueous layer was separated and the organic layer was washed with 100 mL of water (1 volume). The organic layer was concentrated to dryness under reduced pressure to give 129 g of crude compound as brown dense oil. The crude compound was subjected to distillation to give two fractions. Both the fractions were collected at 120 to 130° C. (oil bath temp), 96° C. to 100° C. distillate temperature, 3 mbar vacuum. The first few grams were collected as fraction 1 to give 5.5 g of colorless oil with 90% AUC purity with 3% AUC methyl ester impurity. The rest was collected as fraction 2 to give 116.8 grams of colorless oil with >96% AUC purity with 2.5% AUC methyl ester impurity (94% yield).

### Example 2

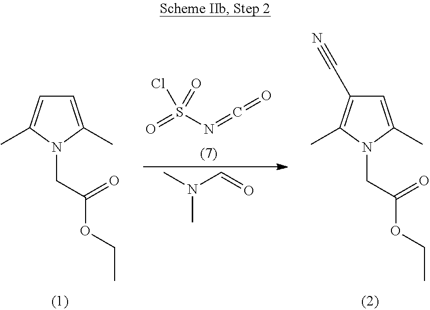

Pyrrole acetic acid ester 1 (345 g, 1904 mmol, 1.0 eq) was dissolved in DMF (2070 mL, 6 vol) at room temperature, in a multineck 5 L round bottom flask, equipped with a mechanical stirrer, an addition funnel and a thermometer, under a nitrogen purge. The flask was then cooled to −64° C. and, under a N₂ atmosphere, and chlorosulfonyl isocyanate (7, CSI, from Aldrich (STBB0539A), 0261 g, 160 mL, 1847 mmol, 0.97 eq), dissolved in acetonitrile (209 mL, 0.8 vol) was added drop-wise, using an addition funnel, at such a rate as to keep the temperature between −64 to −57° C. The reaction was stirred at −60 to −65° C. for 15 minutes and then warmed to −5° C. over 1.5 h. TLC (2:1, hexane/EtOAc, Rf=0.4) and HPLC analyses confirmed completion of the reaction. The mixture was then cooled to −20° C. and quenched between −18 to +14° C. with saturated brine (700 mL), followed by water (350 mL). The reaction mixture was cooled to +8° C. and the solids were filtered, then slurried in water (2 L) for 15 min. Solids were filtered again and rinsed with water (2×1 L) and then dried to constant weight in a vacuum oven at +25° C. (72 hours) to provide 339 g of 2 as a white solid. HPLC analysis showed 98% AUC purity (86% yield).

¹H-NMR (400 MHz, CDCl₃) δ 1.28 (t, J=7.2 Hz, 3H), 2.13 (s, 3H), 2.3 (s, 3H), 4.23 (q, J=7.2 Hz, 2H), 4.48 (s, 2H), 6.05 (s, 1H).

¹³C-NMR (100 MHz, CDCl₃) δ 11.33, 11.85, 14.04, 45.52, 61.98, 90.56, 107.93, 117.34, 129.53, 137.71, 167.5.

HPLC Retention Times (RT)

| Peak | Compound | RT (min.) | RRT |
|---|---|---|---|
| 1 | — | 1.99 | 0.66 |
| 2 | 2 | 3.03 | 1.00 |

### Example 3

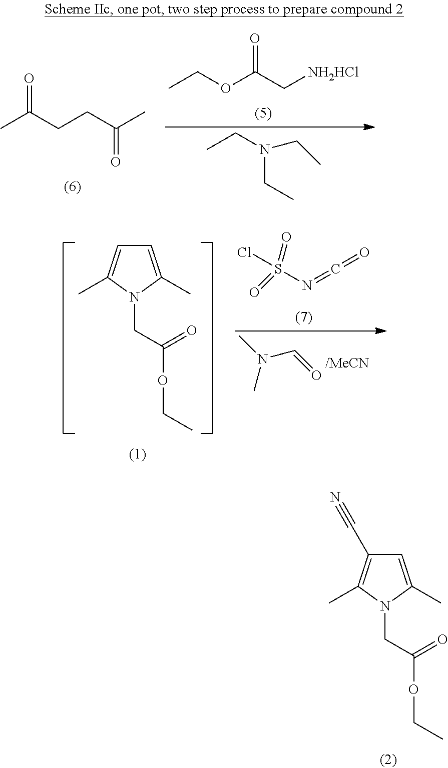

Ethyl 2-aminoacetate hydrochloride (5, 20 g, 143 mmol), hexane-2,5-dione (6, 16.64 ml, 142 mmol, 16.19 g), toluene (120 ml, 6 volumes) and triethylamine (20.37 ml, 146 mmol, 16.19 g) were charged to a clean 250 mL single neck round-bottomed flask equipped with a Dean-Stark apparatus and stirbar and then stirring was initiated. The reaction was refluxed (using an oil bath) at around 115° C. for 4 hours. Approximately 5 mL of water was formed. The color of the reaction looked light yellow and lots of salt formation were observed. The reaction was then allowed to cool to room temperature. The TLC of the reaction mixture confirmed reaction was complete (Hexane:EtOAc, 2:1, KMnO₄ stain, Rf=0.7). 2 volumes of 0.1N HCl (40 mL) was then added to the reaction mixture and this was stirred for 5 mins (clear solution) and then transferred to a separating funnel. The lower brown aqueous layer was separated and the organic layer was washed with water (1×40 and 1×20 mL); then the organic layer was transferred to a 500 mL round-bottomed flask and concentrated using a rotavaporator to give ˜26 gms of a crude brown oil. This was dried under high vacuum for 10 mins. This crude material (1) was directly taken into the next step without any further purification. The crude material was dissolved in DMF (130 ml) and cooled to −67° C. (using a dry ice+acetone bath) and then chlorosulfonyl isocyanate (11.82 ml, 136 mmol, 0.95 eq) in acetonitrile (22.45 ml, 430 mmol, 3 eq) was added drop-wise over 15 mins while maintaining the temperature below −60° C. The reaction mixture was stirred for 15 mins at less than −60° C. and then warmed to −5° C. over 1 hour. HPLC confirmed that the reaction was complete. The reaction mixture was quenched with saturated brine (60 mL, 3 vol based on limiting starting material), followed by water (30 mL, 1.5 vol based on limiting starting material) while controlling the temperature between −5° C. and 10° C. The reaction mixture was stirred for 30 mins. The solids were filtered off and washed with water (60 mL×4), rinsed again with water (20 mL×2) and then dried at 35° C. in the oven for 14 hours to give 22.6 g of product (2) as a white solid. The HPLC of this compound showed a >97% AUC purity with 2% AUC methyl ester impurity.

### Example 4

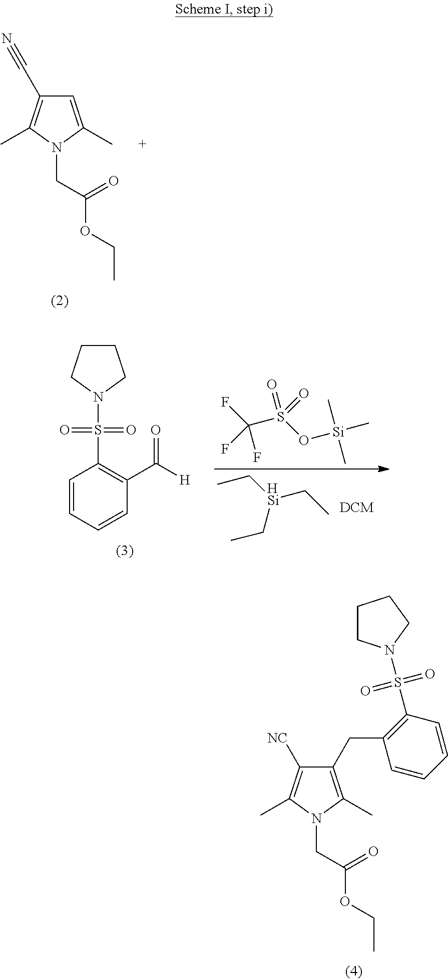

Cyano pyrrole acetic acid ester 2 (65 g, 272 mmol, 1.00 eq) and aldehyde 3 (57.1 g, 277 mmol, 1.02 eq) were charged to a 2 L jacketed reactor, equipped with a mechanical stirrer, an addition funnel and a thermometer, under a nitrogen purge, followed by addition of DCM (650 mL, 10 mL/g). The solution was cooled to −30 to −25° C., and then trimethylsilyl trifluoromethanesulfonate (TMSOTf, from Fluka (0001383669), 98 mL, 543 mmol, 2 eq) was added drop-wise, at such a rate as to keep the temperature between −28 to −23° C. Upon completion of the addition, the reaction was warmed to −17° C. over 90 minutes. HPLC analysis of an aliquot at 90 minutes showed complete disappearance of starting material 2. Therefore, triethylsylane (TES, from Alfa (10152436), 108 mL, 679 mmol, 2.5 eq) was added drop-wise, at such a rate as to maintain a temperature between −17 and −15° C. Upon completion of addition, the temperature was raised to +15° C. over 2 h. HPLC analysis of an aliquot after this time showed the reaction was complete. The reaction was therefore cooled to +8° C. and quenched with aqueous Na₂CO₃ (1.4 eq, 0.5 M, 40 g in 750 mL of water, 11 vol). After completion of the quench, the pH of the aqueous layer was between 9 and 12. The layers were stirred for 10 min and allowed to separate. The aqueous layer was separated and discarded and the organic layer was washed twice with aqueous NaCl (58.4 g, 1 eq in 520 mL of water, 8 vol). The organic layer was concentrated to ⅓ of its original volume (by distillation), then EtOAc (250 mL) was added and the mixture concentrated again to ⅓ of the original volume. This process was repeated. The reaction mixture was then diluted with EtOAc to bring the total volume to 390 mL (6 vol). The resulting solution was heated to +55 to +60° C., and heptanes (500 mL, 7.7 vol) was added drop-wise (using an addition funnel) over 30 minutes. Upon completion of the addition of the antisolvent, ˜20 mg of seeds of cyano pyrrole acetic acid ester 4 (obtained in a previous experiment) were added resulting in a seed bed forming within 5 minutes. The temperature was dropped to +20° C. over 4 h and stirring was continued for 16 h at +20° C. The solids were filtered, washed with a 1:2 mixture of EtOAc/heptanes (300 mL) and dried at +40° C. in a vacuum oven for 24 hours. The desired cyano pyrrole acetic acid ester 4 was obtained as an off-white, tan, solid (101.5 g, 87% yield). HPLC analysis of this material showed a 97.6% AUC purity, with 2% AUC methyl ester impurity.

¹H-NMR (400 MHz, CDCl₃) δ 1.30 (t, J=7.6 Hz, 3H), 1.91-1.94 (m, 4H), 2.0 (s, 3H), 2.33 (s, 3H), 3.32-3.36 (m, 4H), 4.22-4.28 (m, 4H), 4.53 (s, 2H), 7.0 (d, J=7.6 Hz, 1H), 7.30 (t, J=7.2 Hz, 1H), 7.40 (dt, J=1.2 Hz, J=8.0 Hz, 1H), 7.99 (dd, J=1.2 Hz, J=7.6 Hz, 1H)

¹³C-NMR (100 MHz, CDCl₃) δ 9.74, 11.5, 14.1, 25.54, 27.43, 45.74, 47.25, 62.07, 92.92, 116.59, 117.69, 126.15, 127.54, 129.79, 129.86, 132.84, 136.62, 137.17, 139.73, 167.53

HPLC retention times

| Peak | Compound | RT (min.) | RRT |
|---|---|---|---|
| 1 | S-1 | 0.8 | 0.13 |
| 2 | methyl ester | 6.04 | 0.96 |
| 3 | 4 | 6.28 | 1.00 |

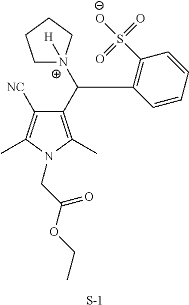

### Example 5

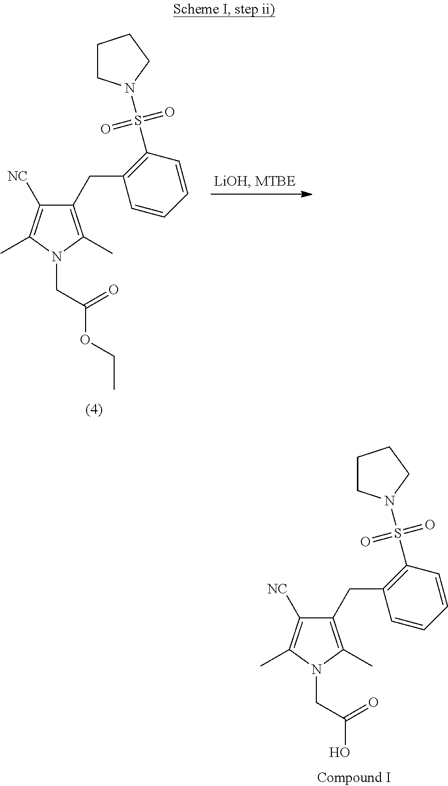 [SMILES: CCOC(=O)Cn1c(C)c(C#N)c(Cc2ccccc2S(=O)(=O)N2CCCC2)c1C ; Cc1c(C#N)c(Cc2ccccc2S(=O)(=O)N2CCCC2)c(C)n1CC(=O)O]

Cyano pyrrole acetic acid ester 4 (176.5 g, 403 mmol, 1.0 eq.) and MTBE (1.41 L, 8.vol) were charged to a 5 L jacketed reactor vessel, equipped with a mechanical stirrer, an addition funnel and a thermometer, under a nitrogen purge. The resulting slurry was stirred at 20° C. In a separate flask, lithium hydroxide (14.5 g, 605 mmol, 1.5 eq) and water (1.06 L 6 vol) were mixed to form a solution. This solution was then added to the reaction mixture with stirring. The reaction was warmed to a temperature between +40 and +45° C. and stirred for 5 h, then cooled to between +25 and +35° C. The basic aqueous layer was transferred back to the cleaned reactor and cooled to <0° C. The aqueous layer was acidified with hydrochloric acid (1 N, 325 mL) at a temperature between −3 and +4° C. until the pH was ˜5, during which time solids formed. Ethyl acetate (1.4 L, 8 vol) was added to dissolve all of the solids. The remaining 1 N HCl (375 mL) was added between +5 and +8° C. until a pH of 2 was achieved. The mixture was stirred for 5 minutes and then the layers were separated. The aqueous layer was extracted with EtOAc (700 mL, 4 vol) and the layers were separated again. The combined organic layers were transferred back to the cleaned reactor and washed with water (700 mL, 4 vol). The organic layer was washed again with water (350 mL, 2 vol). The organic layer was distilled under partial reduced pressure until the volume in the reactor was 1250 mL (7 vol). The remaining solution was cooled to between +24 and +26° C. and seeded (68 mg of Compound I, from a previous experiment). The mixture was stirred at +24 to +26° C. for 0.5 hours then heated to +50 to +55° C. n-Heptanes (1.8 L, 14 vol) was added while maintaining the temperature between +51 and +55° C., during which time the mixture became a thick tan slurry. The slurry was stirred at +55 to +60° C. for 15 minutes, then cooled from +60 to +20° C. over 4 hours and maintained at that temperature for 11 hours. The slurry was cooled to +4 to +6° C. over 1.5 hours then held at this temperature for 3.5 hours. The solids were filtered, washed with ˜1 L of mother liquor, then with 3:1 n-heptanes:EtOAc (1.1 L, 6 vol), and dried in a vacuum oven at +40° C. for 3 days. The desired Compound I with 99.6% AUC purity was obtained as a tan solid (151.8 g, 94% yield).

¹H-NMR (400 MHz, CDCl₃) δ 1.92-1.95 (m, 4H), 2.3 (s, 3H), 2.35 (s, 3H), 3.33-3.36 (m, 4H), 4.29 (s, 1H), 4.61 (s, 2H), 7.0 (d, J=7.2 Hz, 1H), 7.31 (t, J=7.2 Hz, 1H), 7.41 (dt, J=1.6 Hz, J=7.6 Hz, 1H), 7.99 (dd, J=1.6 Hz, J=7.6 Hz, 1H).

¹³C-NMR (100 MHz, CDCl₃) δ 9.76, 11.53, 25.56, 27.43, 45.28, 47.32, 65.01, 93.04, 116.40, 117.95, 126.28, 127.60, 129.82, 129.94, 132.94, 136.55, 137.36, 139.85, 171.53.

HPLC Retention Times (RT) (IPC method)

| Peak | Compound | RT (min.) | RRT |
|---|---|---|---|
| 1 | Compound I | 5.4 | 1.00 |
| 2 | unk. impurity | 5.69 | 1.05 |
| 3 | S-2 | 6.33 | 1.17 |

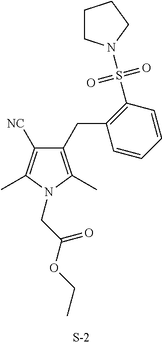

### Example 6

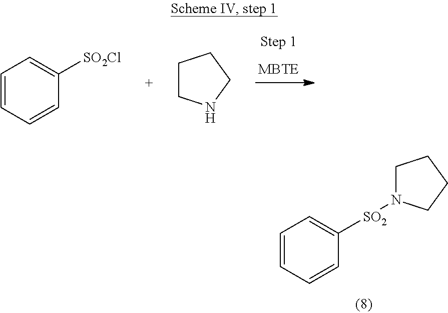 [SMILES: O=S(=O)(Cl)c1ccccc1 ; C1CCNC1 ; O=S(=O)(c1ccccc1)N1CCCC1]

An appropriately sized reactor was charged with pyrrolidine (24.76 kg, 348.2 moles) and MTBE (72 L) and the mixture was stirred until a clear solution was obtained. The reactor was blanked with nitrogen. The temperature of the reaction mixture was adjusted to a value between +5° C. and +10° C. Benzenesulfonyl chloride (30 kg, 169.9 moles) was then added slowly via an addition funnel over a minimum of 1 h, at a rate slow enough to keep the temperature below +45° C. (Note: an extremely strong exotherm was observed; the reaction mixture formed a thick suspension as solids of pyrrolidine hydrochloride started to precipitate). The addition funnel was then rinsed with MTBE (3 L). After the addition of benzenesulfonyl chloride was complete, the reaction mixture was stirred at +35° C. to +45° C. for 30 min. The reaction was monitored by TLC (SiO₂, Ethyl acetate/heptanes [1:9], UV) with the R_{f} of the starting material benzenesulfonyl chloride=0.3 and the R_{f} of the product=0.1. The reaction was deemed complete upon complete visual disappearance of starting material.

Upon completion of the reaction, the temperature of the reaction mixture was adjusted to between +24° C. and +28° C. (Note: avoid cooling to below +20° C., as it may result in partial precipitation of the product). The white solids of pyrrolidine hydrochloride were filtered off by vacuum filtration and washed with MTBE (2×37.5 L, 2×1.25 vol). With stirring, the filtrate was cooled to a temperature between +5° C. and +10° C. and a crystalline suspension formed, which was stirred at +5° C. to 0° C. for a minimum of 15 minutes. Heptanes (150 L, 5 vol) were then added slowly over a minimum of 15 minutes, while maintaining the temperature of the suspension below +10° C. The suspension was stirred at +2° C. to +8° C. for a minimum of 2 hours and the resulting solids were isolated by vacuum filtration, rinsed with heptanes (2×30 L, 2×1.0 vol) and dried under high vacuum at room temperature to a constant weight. The product was obtained as a white crystalline solid (28,247 Kg, 79% yield). The purity as determined by HPLC was 99.98%. ¹H NMR (300 MHz, CDCl₃) δ 7.86-7.83 (m, 2H), 7.64-7.51 (m, 3H), 3.28-3.23 (m, 4H), 1.78-1.73 (m, 4H).

### Example 7

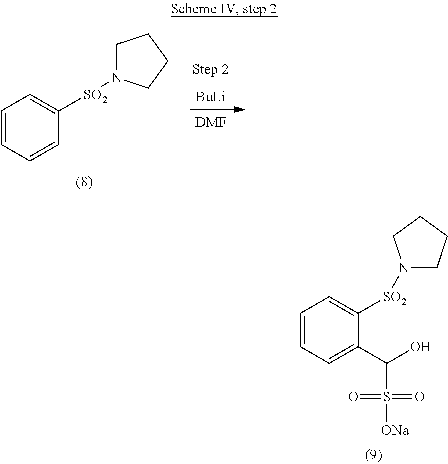

An appropriately sized reactor was charged with tetrahydrofuran (141 L or 125 kg, 5 vol) and (phenylsulfonyl)pyrrolidine (28.25 kg, 133.7 moles). The reaction mixture was cooled to a temperature between −15° C. and −5° C. and, slowly, n-BuLi (58.83 L, 40.6 kg 2.5 M solution in hexanes, 147.1 moles) was added via an addition funnel at a rate as to keep the temperature below 0° C. (Note: a significant exotherm was observed during the addition, which may take more than 1 h to complete. White solids were observed after the addition of n-BuLi). After the addition of BuLi was complete, the reaction mixture was stirred for 1 to 2 hours at a temperature between −5° C. and 0° C. DMF (12.7 kg, 173.8 moles) was then added via an addition funnel at a rate as to keep the temperature below 0° C. (Note: an exotherm was observed, the addition may take more than 30 min to complete). After the addition of DMF was complete, no solids were observed and the reaction mixture was a dark clear solution). The reaction mixture was then allowed to warm-up to ambient temperature over 2 to 3 hours and stirred at +18° C. to +22° C. for a minimum of 1 hour. The reaction was monitored by TLC (SiO₂, ethyl acetate/heptanes[1:1], UV), with the R_{f} of the starting material (phenylsulfonyl)pyrrolidine=˜0.45 and the R_{f} of the product=˜0.4. The reaction was deemed complete when only a trace visual appearance of SM remained. At that point, the reaction mixture was slowly poured into a stirred solution of ammonium chloride (71.5 kg, 10 eq) in water (282.5 kg), while keeping the temperature at between +10° C. and +15° C., followed by stirring for 15 minutes. Agitation was then stopped to allow the phases to separate for a minimum of 5 minutes. The layers were separated and to the aqueous layer was added ethyl acetate (113 L/101.1 kg), followed by stirring for 10 minutes. The aqueous layer was discarded and the organic layers were combined and washed with a solution of ammonium chloride (35.8 kg) in water (141 L/141 kg). The aqueous layer was again discarded and the organic layer concentrated to ˜1.5 to 2 vol (˜43-57 L) under reduced pressure. An oily residue was observed at the end of the concentration with a small amount of white precipitated solid, likely corresponding to inorganic material. This residue was dissolved in DCM (197.75 L, 262.4 kg, 7 vol) at ambient temperature. In a separate reactor, a suspension of sodium metabisulfite (50.84 kg, 267.4 moles) in water (39.55 L, 39.55 kg, 1.4 vol) was prepared and the DCM organic solution obtained above was added to this aqueous sodium metabisulfite suspension. The resulting mixture was agitated for a minimum of 16 hours at ambient temperature (+15° C. to +30° C.). Soon after the DCM solution had been added to the sodium metabisulfite suspension, product precipitation was observed. The solids were collected by filtration and rinsed with DCM (2×56.5 L, 2×75 kg, 2×2 vol). The product was dried under high vacuum at room temperature until all the DCM was removed. The product, containing water and sodium metabisulfite, was obtained as a white solid (72.9 Kg total, corrected amount 45.91 Kg) and used directly in the next step without further purification.

¹H-NMR (400 MHz, DMSO-d₆) δ 1.70-1.74 (m, 4H), 3.09-3.15 (m, 2H), 3.24-3.30 (m, 2H), 6.05 (s, 2H), 7.43 (t, J=7.2 Hz, 1H), 7.57 (t, J=7.2 Hz, 1H), 7.78 (d, J=7.6 Hz, 1H), 8.13 (d, J=8 Hz, 1H).

### Example 8

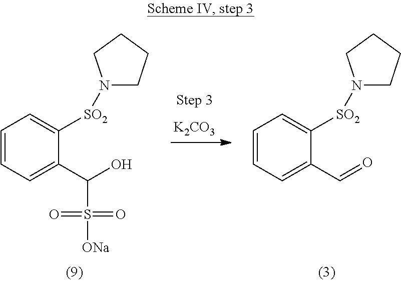

An appropriately sized reactor was charged with water (230 kg, 5 vol) and the crude sodium hydroxyl[2-(pyrrolidinylsulfonyl)-phenyl]methanesulfonate obtained in Example 6 (net amount ˜45.91 kg, 133.7 moles), and stirring initiated. A white suspension formed. Potassium carbonate (110.9 kg, 802.2 moles, 6 equiv) was then added portionwise. As significant foaming was observed, the initial portion sizes should be around 2-3 kg in size, and they can be significantly increased in size as the reaction progresses. The reaction mixture formed a thick slurry of fine solids and the volume of the reaction mixture increased to ˜900-950 L (up to ˜250 gal) due to the foaming. When all excess unreacted sodium metabisulfite present in the starting material had been consumed by potassium carbonate, a reduction of the reaction volume was observed. At this point, the reaction formed a yellow suspension. A moderate exotherm may be observed from +12° C. to +29° C. during this operation but external temperature control was not required. The walls of the reactor were rinsed with water (keeping the volume to a minimum) and stirring was continued at ambient temperature (+18° C. to +22° C.) for a minimum of 16 hours. The solids were then collected by filtration and rinsed with water (34.4 L, 0.75 vol). The product was slurried (in the reactor or on a filter) with water (45.9 L, 1 vol) and the solids collected by filtration, rinsed with water (34.4 L, 0.75 vol) and dried under high vacuum at room temperature until a constant weight was achieved.

The product was obtained as white solids (23.1 Kg, 71%) and stored in a cold room or freezer under nitrogen atmosphere once the weight was constant. The purity as determined by HPLC was 99.86%.

¹H-NMR (400 MHz, CDCl₃) δ 1.76-1.80 (m, 4H), 3.18-3.21 (m, 4H), 7.63-7.69 (m, 2H), 7.86-7.88 (m, 1H), 7.96-7.98 (m, 1H), 10.77 (s, 1H).

¹H NMR (300 MHz, CDCl₃) δ 10.88 (d, J=0.3 Hz, 1H), 8.10-8.07 (m, 1H), 8.01-7.98 (m, 1H), 7.74-7.71 (m, 2H), 3.32-3.27 (m, 4H), 1.90-1.86 (m, 4H).

¹³C-NMR (100 MHz, CDCl₃) δ 25.34, 47.52, 129.06, 129.23, 132.95, 133.43, 134.29, 139.10, 190.80.

1. A process for making a compound of Formula AII, said process comprising the steps of:
i) reductively alkylating a pyrrole starting material 2A with an aldehyde starting material 3A, in the presence of a Lewis acid and a reducing agent, in an aprotic organic solvent to afford an ester of Formula AI, 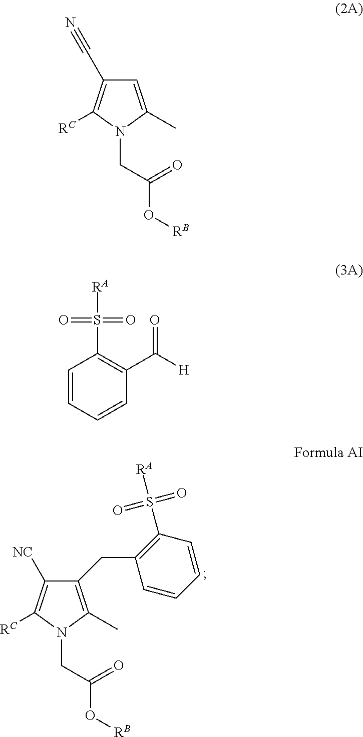
and
ii) saponifying the ester moiety of the ester of Formula AI in a solvent system, in the presence of an alkali metal hydroxide, affording, after treatment with an aqueous mineral acid, an acid of Formula AII, 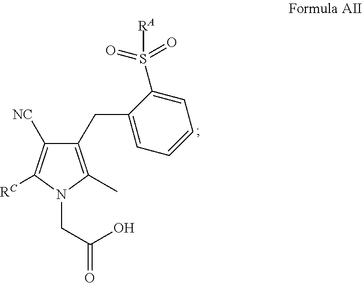
wherein:
R^{A} is selected from phenyl or an N-linked 5 or 6-membered heterocycle, optionally containing up to two other ring heteroatoms selected from O, N or S in addition to the N ring atom of the 5 or 6-membered heterocycle linked to the sulfur atom of the sulfonyl group; wherein, the linked N is directly attached to the sulfur atom of the sulfonyl group to form a sulfonamide;
R^{B} is a C₁₋₆ alkyl; and
R^{C} is selected from C₁₋₆ alkyl, a 3 to 6-membered cycloalkyl ring or phenyl; in said compounds of Formulae 2A, 3A, AI and AII.

2. The process of claim 1, wherein R^{A} is the N-linked 5 or 6-membered heterocycle.

3. (canceled)

4. The process of claim 1, wherein R^{B} is a C₁₋₆ alkyl in said compounds of Formula 2A and Formula AI.

5. (canceled)

6. (canceled)

7. The process of claim 1, wherein R^{C} is selected from C₁₋₆ alkyl, a 3 to 6-membered cycloalkyl ring or phenyl, in said compounds of Formula 2A, Formula AI and Formula AII.

8. (canceled)

9. (canceled)

10. The process of claim 1, wherein the compound of Formula AII is Compound I:

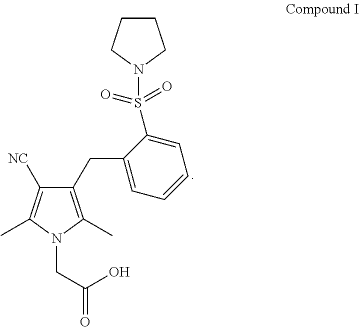 [SMILES: Cc1c(C#N)c(Cc2ccccc2S(=O)(=O)N2CCCC2)c(C)n1CC(=O)O]

11-13. (canceled)

14. The process of claim 1, wherein the temperature used in step i) is between −28° C. and 0° C.

15. (canceled)

16. The process of claim 1, wherein the Lewis acid used in step i) is selected from trifluoromethylsulfonyltrimethylsilane, boron trifluoride etherate or boron trifluoride acetonitrile complex.

17. (canceled)

18. The process of claim 1, wherein the reducing agent used in step i) is a silane.

19. (canceled)

20. (canceled)

21. (canceled)

22. (canceled)

23. (canceled)

24. (canceled)

25. The process of claim 1, wherein the strong inorganic base used in step ii) is an alkali metal hydroxide.

26. (canceled)

27. (canceled)

28. The process of claim 1, wherein, in the saponification reaction in step ii), the solvent system is comprised of a mixture of water and an aprotic solvent.

29. (canceled)

30. (canceled)

31. The process of claim 1, wherein the solvent system used in step ii) additionally comprises an alcohol.

32. (canceled)

33. The process of claim 1, wherein the number of equivalents of base used in step ii) is at least 1.0 equivalents of base per equivalents of the compound of Formula AI.

34. (canceled)

35. (canceled)

36. The process of claim 1, wherein saponification step ii) is conducted at reflux.

37-65. (canceled)

66. A process for preparing ethyl 2,5-dimethyl-3-cyano-pyrrol-1-ylacetate comprising:
A) reacting ethyl 2-aminoacetate hydrochloride with hexane-2,5-dione in toluene, in the presence of an organic base at reflux;
B) quenching the product of step A) with an aqueous mineral acid;
C) separating the organic layer of the product of step B);
D) washing the organic layer with water;
E) concentrating the washed organic layer to give a crude oil containing ethyl 2,5-dimethyl-pyrrol-1-ylacetate;
F) dissolving ethyl 2,5-dimethyl-pyrrol-1-ylacetate in the crude oil obtained in step E) in DMF without any purification or isolation of ethyl 2,5-dimethyl-pyrrol-1-ylacetate, cooling the DMF solution, and reacting ethyl 2,5-dimethyl-pyrrol-1-ylacetate with chlorosulfonyl isocyanate dissolved in acetonitrile to obtain ethyl 2,5-dimethyl-3-cyano-pyrrol-1-ylacetate.

67. The process of claim 66, further comprising
G) quenching the product of step F) with brine, followed by water, while controlling the temperature between −5° C. and 10° C.;
H) filtering off the solids from the product of step G);
I) washing the filtered solids with water; and
J) drying the washed solids to yield solid ethyl 2,5-dimethyl-3-cyano-pyrrol-1-ylacetate.

68. The process of claim 66, wherein the organic base used in step A) is selected from triethylamine, diethylamine, Huenigs base, pyridine, diisopropylethylamine or mixtures thereof.

69. (canceled)

70. The process of claim 66, wherein the aqueous mineral acid used in step B) is diluted aqueous acid.

71. (canceled)

72. (canceled)

73. (canceled)

74. A process for the synthesis of compound 3, comprising the steps of:
I) reacting benzylsulfonyl chloride with pyrrolidine, in an aprotic organic solvent to furnish compound 8; 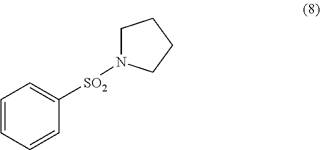 [SMILES: O=S(=O)(c1ccccc1)N1CCCC1]
II) generating a carbanion alpha to the sulfonyl group of the phenyl ring of compound 8, by using a strong organic base, in an aprotic organic solvent, at a low temperature between −78° C. and 0° C., and quenching said carbanion with a carbaldehyde donor, to furnish an hydroxy(2-(pyrrolidin-1-ylsulfonyl)phenyl)methanesulfonate of formula 9; and 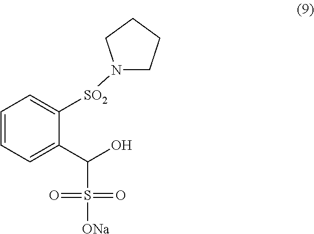
III) treating hydroxy(2-(pyrrolidin-1-ylsulfonyl)phenyl)methanesulfonate of formula 9 with an inorganic base, in a solvent system to provide compound 3:

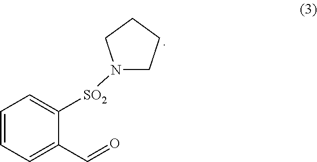 [SMILES: O=Cc1ccccc1S(=O)(=O)N1CCCC1]

75. The process of claim 74, wherein the aprotic organic solvent used in step I) is selected from: tetrahydrofuran, methyl tert-butyl ether, dioxane, acetone, 2-butanone, dichloromethane, dichloroethane or chloroform.

76. (canceled)

77. (canceled)

78. The process of claim 74, wherein the strong organic base used in step II) is selected from n-BuLi, s-BuLi, t-BuLi, iPrMgCl, LDA or LiHMDS.

79. (canceled)

80. The process of claim 74, wherein the aprotic organic solvent used in step II) is selected from: THF, dioxane, N,N-dimethylformamide, N,N-dimethylacetamide, N-methylpyrrolidine or 1,3-dimethyl-2-imidazolinone.

81. (canceled)

82. (canceled)

83. (canceled)

84. The process of claim 74, wherein the inorganic base used in step III) is selected from: sodium carbonate or potassium carbonate.

85. (canceled)

86. The process of claim 74, wherein the solvent system used in step III) comprises a mixture of water and an organic solvent.

87. (canceled)

88. (canceled)

89. The process of claim 74, wherein the solvent system used in step III) consists of water.

90. (canceled)

91. (canceled)

92. A compound of Formula AI or AII, or a pharmaceutically acceptable salt thereof, 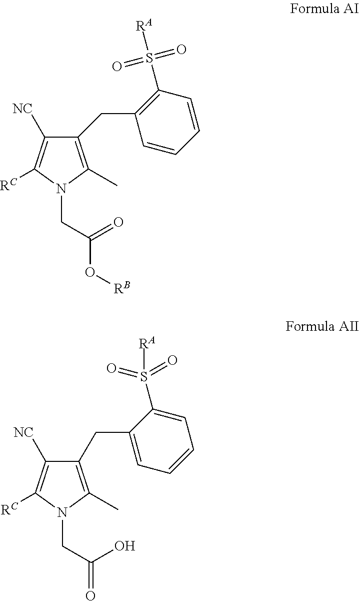
wherein:
R^{A} is selected from phenyl or an N-linked 5 or 6-membered heterocycle, optionally containing up to two other ring heteroatoms selected from O, N or S in addition to the N ring atom of the 5 or 6-membered heterocycle linked to the sulfur atom of the sulfonyl group; wherein, when R^{A} is an N-linked heterocycle, the linked N is directly attached to the sulfur atom of the sulfonyl group to form a sulfonamide;
R^{B} is a C₁₋₆ alkyl; and
R^{C} is selected from C₁₋₆ alkyl, a 3 to 6-membered cycloalkyl ring or phenyl.

93. The compound of claim 92 having Formula AII, or a pharmaceutically acceptable salt thereof.

94. The compound of claim 93 having the following formula, or a pharmaceutically acceptable salt thereof 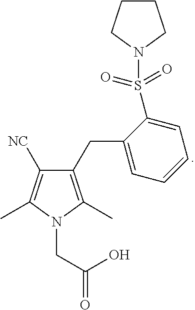 [SMILES: Cc1c(C#N)c(Cc2ccccc2S(=O)(=O)N2CCCC2)c(C)n1CC(=O)O]
